(19) United States
(12) Patent Application Publication
(10) Pub. No.: US 2004/0192896 A1
(43) Pub. Date: Sep. 30, 2004

(54) Method for extracting gluten

(75) Inventor: Stephen Patrick Finch, New London, WI (US)

Correspondence Address: Robert O. Blinn, P.O. Box 75144, Wichita, KS, 67275-0144, US

(73) Assignee: ICM, Inc., Colwich, KS

(21) Appl. No.: 10/757,246

(22) Filed: Jan. 14, 2004

Related U.S. Application Data
(60) Provisional application No. 60/439,970, filed on Jan. 14, 2003.

(51) Int. Cl. C07K 14/415

(52) U.S. Cl. 530/374

(57) ABSTRACT

The present invention is a method for separating gluten from wheat flour to produce a protein isolate that is at least 90% protein. The method includes at least one emulsification step where the starch within gluten masses is exposed. Masses composed of mostly gluten and some starch from a typical agglomeration tank and screen rinse process are processed through at least one emulsifying step where the masses are chopped and then drawn through a perforated plate to produce relatively small gluten masses. The relatively small gluten masses from the at least one emulsifying step are screen rinsed to remove much of the remaining starch thus producing a protein isolate. The resulting protein isolate gluten product is then dried and processed using conventional methods known in the art.

### CROSS REFERENCE TO RELATED APPLICATIONS

This application claims the benefit of U.S. Provisional Patent Application No. 60/439,970 filed Jan. 14, 2003.

### FIELD OF THE INVENTION

The present invention relates to a method for extracting gluten from grain flour.

### BACKGROUND OF THE INVENTION

The separation of wheat flour into its component parts, primarily starch and gluten, has been practiced at least as early as the first half of the nineteenth century. A number of methods have been devised for separating starch and gluten. The Martin method is a commonly used industrial process which includes the steps of forming a dough by mixing wheat flour with a small amount of water, allowing the dough to rest or mature, kneading the dough while adding water and gradually washing off the starch from the gluten. The Fesca process includes the steps of making a wheat flour slurry and centrifuging that slurry to separate the starch from the gluten. In a method taught by Kerkkonen et al. in U.S. Pat. No. 3,951,938, a suspension of flour in water is centrifuged into a heavy portion and a light gluten rich portion which is then mixed with water, allowed to agglomerate and then is separated using vibrating screens. Verbeme et al. in U.S. Pat. No. 4,132,566 teach a process in which a mixture containing wheat protein and water is treated in a hydrocyclone apparatus.

Many of the prior art methods that are typically practiced produce a protein “concentrate” having a purity of approximately 75%. Demand exists however for gluten “protein isolates” having protein concentrations above 90%. One commonly practiced method for producing a protein isolate is to add an acid such as hydrochloric acid to a protein concentrate to dissolve a significant portion of the starch present in the concentrate. After much of the starch has been removed, the acid remaining with the protein isolate is neutralized with a hydroxide such as sodium hydroxide. However, a residue of salt remains with the protein isolate after the remaining acid is neutralized. Such a salt residue will interfere with the taste of the resulting protein isolate. Ideally, a protein isolate, which may be used in a number of food products, should have a bland taste that does not interfere with the taste of the food product.

### SUMMARY OF THE INVENTION

The present invention is a method for separating gluten from wheat flour without using an acid. Because no acids are used, the gluten protein isolate produced using this method has no salt residue. The method of the present invention includes a well known gluten agglomeration step followed by a screen washing step where starch is washed away from gluten agglomerates to produce masses composed mostly of gluten having gluten concentrations of approximately 70%. In order to remove much of the remaining starch from the masses composed mostly of gluten, the masses are processed through at least one emulsification step. In the preferred embodiment the masses are first processed through a batch emulsification step and then processed through a continuous emulsification step. In the batch emulsification step, the gluten masses are forced in a repeating fashion through a passage having rotating blades and a perforated plate. The pulverizing action of the batch emulsification step produces much smaller gluten masses. The relatively small gluten masses from the batch emulsification step are stored in a surge storage tank which continuously feeds a screen rinse unit where some of the remaining starch is rinsed away from the gluten masses to produce small masses of relatively high gluten content. The small masses of relatively high gluten content from the screen rinse step are then delivered to the continuous emulsifier where the masses are again chopped by a rotating blade and drawn through a perforated plate. The masses leaving the second emulsifier are rinsed in a rotary drum rinse unit to strip away more of the remaining starch from the gluten to produce gluten masses. The resulting gluten masses are at least 90% protein and are therefore a protein isolate. They are also substantially free of any salt or chemical residues. The resulting protein isolate is then pressed and dried and processed into a dry powder using conventional methods known in the art.

### BRIEF DESCRIPTION OF THE DRAWINGS

FIG. 1 is a flow chart of the method of the present invention.

FIG. 2 is a schematic diagram of the method of the present invention.

### DETAILED DESCRIPTION OF THE INVENTION

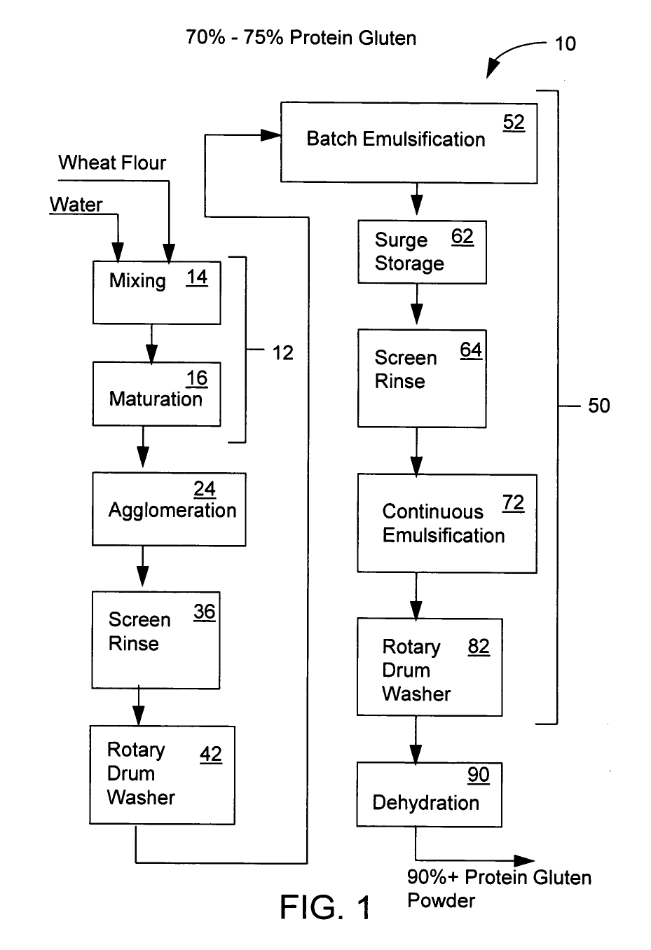

Turning now to the drawings and more particularly FIG. 1 thereof, wherein like reference numerals identify identical or corresponding process steps, the gluten extraction method 10 is shown generally including a mixing portion 12, an agglomeration portion 24, a screen rinse portion 36, and an emulsification portion 50.

Mixing portion 12 is a typical process where flour is combined with water and an protein starch breaking agent and matured in order to remove starch and retain gluten. Mixing portion 12 generally includes a mixing step 14 where wheat flour is mixed with water and an agent for breaking protein starch bonds. Typically, an enzyme or a salt may used as an agent for breaking protein starch bonds. In this process, it is preferable to use an enzyme. In maturation step 16, the resulting dough is allowed to rest for a length of time.

Agglomeration portion 24 is accomplished in a generally horizontal agglomeration tank 26 which includes an outer water tight shell and agitators which move the masses of dough from one end of agglomeration tank 26 to the other end as water is added and as gluten in the dough agglomerate into to masses composed mostly of gluten.

Screen rinse portion 36 is best performed using screens oriented at an angle of substantially 45 degrees. Screen rinse portion 36 preferably includes a series of screen rinse units which process the gluten masses in a sequential manner. Gluten masses from agglomeration tank 26 tumble down the screens of the screen rinse units of screen rinse portion 36 as the masses are sprayed with warm water. This action rinses much of the starch away from the gluten masses. Further washing is accomplished in a rotary drum washer 42. After screen rinse portion 36 and rotary drum washer 42, the gluten masses should have a protein concentration of approximately 70% to 75%.

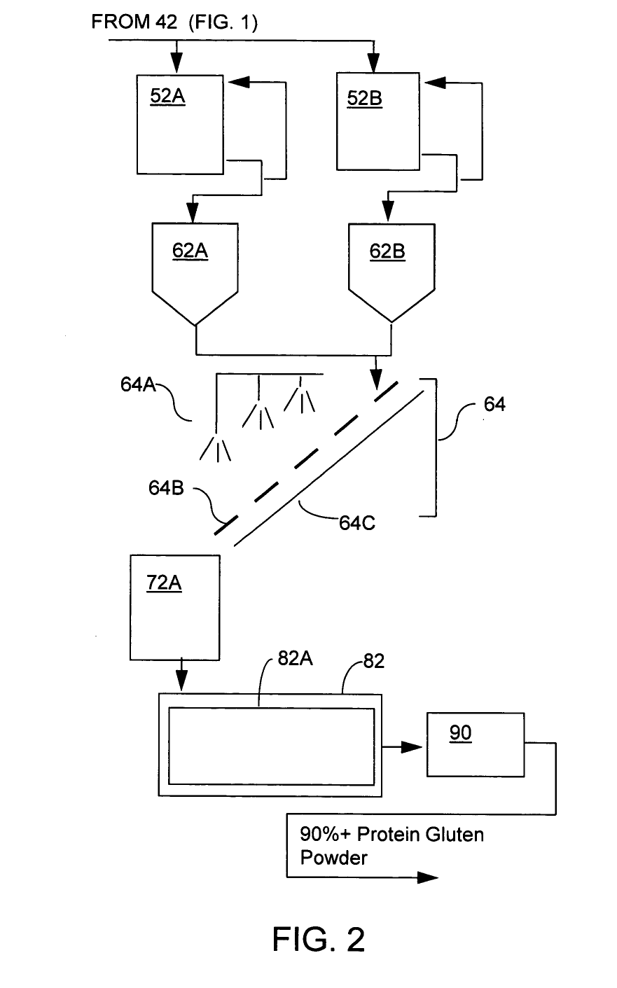

Emulsification portion 50 receives gluten masses consisting of approximately 70% to 75% gluten from rotary drum washer 42 and produces gluten masses having a protein concentration in excess of 90%. Emulsification portion 50 includes a batch emulsification step 52, a surge storage step 62, a screen rinse step 64, a continuous emulsification step 72 and a rotary drum screen rinse step 82. The purpose of the emulsification steps of emulsification portion 50 is to disrupt bonds that exist between starch and protein molecules within the gluten masses, expose the resulting unattached starch molecules so that the exposed starch molecules may be rinsed away during the rinsing steps that occur immediately following each emulsification step. Preferably, the items of equipment of steps 52 and 62 are duplicated as two sets of equipment so that the each of the two sets of equipment may be fed in a continuous yet alternating fashion from screen rinse portion 36. The two surge tanks 62A and 62B shown in FIG. 2 of surge storage step 62 may then be drawn from in an alternating fashion to feed screen rinse unit 64 in a substantially continuous fashion. As is diagrammed in FIG. 2, screen rinse unit 64 includes a water spraying apparatus 64B and an inclined screen 64C. Gluten masses proceed from the top edge to the lower edge of screen 64C and are fed into continuous emulsifier 72A.

Both batch emulsification step 52 and continuous emulsification step 72 are performed using emulsifying machines that employ rotating blades in combination with perforated annular plates. Generally, these emulsifying machines pull agglomerated gluten masses through a cylindrical throat that leads to a zone where the masses encounter a rotating blade and a perforated annular plate. The gluten masses first encounter the rotating blade which has blade edges set in close proximity to the surface of the perforated annular plate. The blades cut gluten masses into smaller masses. The machine draws these smaller masses through the holes in the perforated plate. The resulting gluten masses are much smaller than the gluten masses entering the machine and have been greatly disrupted such that protein starch bonds are broken.

In batch emulsification step 52, a batch of gluten masses are processed through a batch emulsifying machine 52A or 52B shown in FIG. 2 so that starch protein bonds are disrupted throughout the material. This may be done in a once through manner or in a repetitive manner. Batch emulsification machines 52A and 52B can be set to run a batch of gluten in a cyclic or repetitive manner such that one batch of gluten is recycled and thus emulsified by emulsification machine 52A or 52B more than once. These repetitive cycles increases the degree by which protein is separated from the remaining starch. Accordingly, increasing the number of cycles of emulsification in this step will increase the concentration of the protein isolate yielded at the end of the process. Preferably, batch emulsification machines 52A and 52B shown in FIG. 2 are Karl Schnell Model B-22, type 250I emulsifiers. Karl Schell emulsifiers are distributed by Karl Schnell, Inc. of New London, Wis.. Machines of this general type are also often referred to as comminuting machines

Once the gluten masses has been emulsified in batch emulsification step 52, they are immediately washed in screening step 62 that employs a screen rinse unit 64. Screen rinse unit 64 is shown schematically in FIG. 2. It is of a type having a flat inclined screen 64B which has a fine mesh size between 60 and 130 microns. The gluten masses from batch emulsification step 52 are deposited at the upper edge of screen 64B and are continuously sprayed with warm water as they proceed toward the lower edge of inclined screen 64B. The gluten masses leaving screen rinse unit 64 may re-agglomerate into larger masses.

The rinsed gluten masses from screen rinse unit 64 are then fed in a continuous manner into continuous emulsification step 72. Continuous emulsification step 72 is conducted by a continuous emulsification machine 74 shown schematically in FIG. 2. Continuous emulsification machine 74 also includes a passageway, rotating blades and a perforated plate. Gluten masses are conducted through the passageway, across the rotating blade and through the perforated plate in a continuous, once-through manner. Preferably, continuous emulsification machine 72A is a Karl Schnell Model FD2 emulsifier. Once gluten has been pulverized in continuous emulsification step 72, it is immediately washed in rotary drum washer 82.

The gluten masses leaving rotary drum washer 82 are substantially free of starch and should consist of at least 90% gluten protein. The gluten product of rotary drum washer 82 may be processed in any number of ways known by those skilled in the art. Typically, the product will be pressed to remove water and then dried using methods well known in the art in a dehydration step 90. The dried gluten can be processed into a powder.

Accordingly, the method of the present invention meets the objectives described above by providing a way to extract gluten from wheat flour to present a wheat gluten product that is substantially free of starch and which consists of at least 90% gluten protein.

The skilled reader, in view of this specification may envision numerous modifications and variations of the above disclosed preferred embodiment.

Accordingly, the reader should understand that these modifications and variations, and the equivalence thereof, are within the spirit and scope of this invention as defined in the following claims.

Having thus described the invention, what is claimed as new and desired to be secured by Letters Patent is:

1. A method for extracting gluten from flour comprising the following steps:
(a) mixing milled flour with water and an agent for breaking chemical bonds between starch molecules and protein molecules,
(b) maturing the flour and water mixture in a maturation tank,
(c) combining the flour and water mixture into masses of dough consisting mostly of gluten,
(d) presenting the masses of dough to at least one rinser having a screen for retaining the masses of dough as starch is rinsed away from the masses composed mostly of gluten,
(e) feeding the rinsed masses composed mostly of gluten produced in step (d) through a comminuting machine which forces the dough through a channel wherein the dough alternately encounters at least one set of cutting blades and at least one perforated plate to produce smaller masses composed mostly of gluten,
(f) presenting the smaller masses composed mostly of gluten produced in step (e) to at least one rinser having a screen for retaining the smaller masses composed mostly of gluten as a substantial portion of the remaining starch is rinsed away from the smaller masses,
(g) drying the rinsed smaller masses produced in step (f) to produce a dry product consisting mostly of gluten protein.

2. The method of claim 1, further comprising the following steps:
a first additional step wherein the product of step (f) is allowed to recombine into larger recombined masses, and wherein steps (e) and (f) are performed again on the recombined masses from the first additional step.

3. The method of claim 1, wherein:
step (e) is replaced by the folowing steps:
(i) a first emulsification step where the masses from step (d) are fed through a first comminuting machine in batches to produce emulsified gluten masses,
(ii) rinsing the emulsified gluten masses from step (i) and,
(iii) feeding the rinsed emulsified gluten masses from step (ii) through a second emulsification step where the rinsed emulsified gluten masses are further emulsified.

4. A method for separating gluten from grain flour to produce a protein isolate, comprising the following steps:
(a) mixing grain flour with water and a protein starch bond breaking agent to produce masses of dough,
(b) agglomerating the masses of dough by moving the masses of dough of step (a) through an agglomeration tank wherein a portion of the starch in the dough is washed away from the masses to produce masses composed mostly of gluten,
(c) rinsing the masses composed mostly of gluten of step (b) with water over a screen to produced rinsed gluten masses,
(d) emulsifying the rinsed gluten masses of step (c) by passing the rinsed gluten masses through a throat and past rotating blades to produce chopped gluten masses and then drawing the chopped gluten masses through a perforated plate proximate to the rotating blades to produce relatively small chopped gluten masses,
(e) rinsing the relatively small chopped gluten masses from step (d) to remove a portion of the remaining starch such that the resulting gluten masses are at least 90% gluten thereby providing masses of protein isolate.

5. The method of claim 4 wherein,
the emulsification step of step (d) includes the following steps
(i) a batch emulsification step in which batches of rinsed gluten masses from step (c) are passed through a throat, past rotating blades and through a perforated plate, and
(ii) a continuous emulsification step in which rinsed emulsified gluten masses from step (i) are passed through a throat, past rotating blades and through a perforated plate in a continuous manner.

6. The method of claim 4 wherein,
the emulsification step of step (d) includes the following steps
(i) a batch emulsification step in which batches of rinsed gluten masses from step (c) are passed through a throat, past rotating blades and through a perforated plate at least two times, and
(ii) a continuous emulsification step in which rinsed emulsified gluten masses from step (i) are passed through a throat, past rotating blades and through a perforated plate in a continuous manner.

7. The method of claim 4 wherein,
the emulsification step of step (d) includes the following steps
(i) a batch emulsification step in which rinsed gluten masses from step (c) are passed through a throat, past rotating blades and through a perforated plate in a repetitious manner,
(ii) a surge storage step where emulsified gluten masses from step (i) are stored in a surge storage tank,
(iii) a rinsing step in which emulsified gluten masses are drawn from the surge storage tank of step (ii) and rinsed with water,
(iv) a continuous emulsification step in which rinsed emulsified gluten masses from step (iii) are passed through a throat, past rotating blades and through a perforated plate in a once through, continuous manner, and,
(v) a washing step where the gluten masses from step (iv) are rinsed of substantially most of their remaining starch.

8. The method of claim 4 wherein,
the emulsification step of step (d) includes the following steps
(i) a batch emulsification step in which rinsed gluten masses from step (c) are passed through a throat, past rotating blades and through a perforated plate in a repetitious manner,
(ii) a surge storage step where emulsified gluten masses from step (i) are stored in a surge storage tank,
(iii) a screen rinse step in which emulsified gluten masses are drawn from the surge storage tank of step (ii) and passed over a screen while rinsed with water,
(iv) a continuous emulsification step in which rinsed emulsified gluten masses from step (iii) are passed through a throat, past rotating blades and through a perforated plate in a once through, continuous manner, and,
(v) washing step where the gluten masses from step (iv) are rinsed of substantially most of their remaining starch.

9. A method for separating gluten from grain flour to produce a protein isolate, comprising the following steps:
(a) mixing grain flower with water and a protein starch bond breaking enzyme to produce masses of dough,
(b) agglomerating the masses of dough by moving the masses of dough of step (a) through an agglomeration tank wherein a portion of the starch in the dough is washed away from the masses to produce masses composed mostly of gluten,
(c) rinsing the masses composed mostly of gluten of step (b) with water over a screen to produced rinsed gluten masses,
(d) washing the masses composed mostly of gluten in a rotary drum washer,
(e) emulsifying the whshed gluten masses of step (d) by
(i) emulsifying the rinsed gluten masses in a batch emulsification step in which rinsed gluten masses from step (d) are passed through a throat, past rotating blades and through a perforated plate,
(ii) storing the gluten masses from step (i) in a surge storage tank,
(iii) rinsing the gluten masses from the surge storage tank,
(iv) providing a substantially continuous supply of gluten masses from the surge storage tank to a continuous emulsification step in which rinsed emulsified gluten masses from step (iii) are passed through a throat, past rotating blades and through a perforated plate in a once through, continuous manner, and,
(e) washing the relatively small chopped gluten masses from step (d) (iv) to remove a portion of the remaining starch such that the resulting gluten masses are at least 90% gluten thereby providing masses of protein isolate.

10. The method of claim 9 wherein,
batch emulsification step (e)(i) is repeated more than once with each batch of masses to increase the resulting protein concentration.
